(19) United States
(12) Patent Application Publication
(10) Pub. No.: US 2025/0109086 A1
(43) Pub. Date: Apr. 3, 2025

(54) METHANOL LOOP REVAMP BY CO INTENSIFICATION

(71) Applicant: Topsoe A/S, Kgs. Lyngby (DK)

(72) Inventors: Peter Mølgaard MORTENSEN, Roskilde (DK); John Bøgild HANSEN, Humleæk (DK); Mathias JØRGENSEN, Hillerød (DK); Søren Grønborg ESKESEN, Espergærde (DK)

(73) Assignee: Topsoe A/S, Kgs. Lyngby (DK)

(21) Appl. No.: 18/834,483

(22) PCT Filed: Apr. 3, 2023
(86) PCT No.: PCT/EP2023/058637

§ 371 Date: Jul. 30, 2024

(30) Foreign Application Priority Data
Apr. 8, 2022 (EP) 22167365.0

(51) Int. Cl. C07C 29/151; B01D 53/00; B01J 19/00; B01J 19/24; C01B 3/02; C07C 29/152

(52) U.S. Cl. CPC C07C 29/1518; B01D 53/002; B01J 19/0013; B01J 19/245; C01B 3/02; C07C 29/152; B01J 2219/00024; B01J 2219/00103

(57) ABSTRACT

A methanol plant is provided in which a first feed comprising CO₂ and a second feed comprising H₂ are provided, the combined feed is shifted in a CO₂ shift unit so as to provide a first synthesis gas stream. Water is removed in a water removal unit, to provide a second synthesis gas stream. A methanol synthesis section receives the second synthesis gas stream, and provides a product stream comprising methanol. A process for producing a product stream comprising methanol, and a process for upgrading a methanol plant are also provided.

### TECHNICAL FIELD

The present invention relates to a methanol plant with improved utilisation of feed gas. A process for producing a product stream comprising methanol, and a process for upgrading a methanol plant are also provided.

### BACKGROUND

Producing methanol from mixtures of CO₂ and H₂ is facing increased focus due to climate issues, and an effort to bring CO₂ by-products from other processes into use.

A straightforward route for methanol production is to mix CO₂ with H₂ directly and react this in a methanol loop (or similar methanol synthesis process technology) according to the reaction:

CO₂+3H₂<=>CH₃OH+H₂O

Herein referred to as CO₂-to-MeOH.

The downside of this process scheme is the significant by-production of water. More conceptually, the methanol reaction is influenced by the actual concentration of CO and CO₂ in the synthesis gas. Optimally, the feed to the methanol synthesis section should only contain 2-3% CO₂. High amounts of CO₂ will result in an increased production of steam because of need to shift the CO₂ to CO in the methanol synthesis reactor, giving an overall lower reaction rate and a higher degree of catalyst deactivation by sintering. When only CO₂ is available as the carbon feedstock, this problem is increased. The consequence is that the methanol synthesis section needs to be relatively big (in terms of reactor volumes, compressor throughput, and other equipment), as the methanol synthesis section practically has to accommodate both reverse water gas shift and methanol synthesis (catalyzed by the same catalyst system).

It is an object of embodiments of the invention to provide a methanol plant in which existing equipment can be used for production of more synthesis gas. This allows for maximizing the output of an already-existing methanol plant. Additionally, the invention has the potential for reducing the relative production price for methanol of the plant.

### SUMMARY

It has been found by the present inventor(s) that improvements in methanol production, and in a methanol plant itself can be achieved by changes upstream the compressor. The invention is based on the recognition that an electrically heated RWGS reactor can be operated at a higher temperature than a fired RWGS reactor, and that hence the RWGS reaction can be shifted to form more CO and water. Hence the use of an electrically heated RWGS reactor makes it possible to reduce the amount of synthesis gas, which is required to be compressed and moved through the plant by the compressor located between the water removal unit and the methanol synthesis section. This in turn reduces the electricity requirement of the compressor and reduces operational costs while making it more feasible to operate the compressor using sustainable electricity. Also, the use of an electrically heated RWGS reactor makes it possible to operate the RWGS reactor on sustainable electricity rather than using a hydrocarbon fuel for a fired RWGS reactor, hence avoiding CO₂ emissions from the plant.

Accordingly, the present technology provides a methanol plant, said methanol plant comprising:
a first feed comprising CO₂ to said plant,
a second feed comprising H₂ to said plant,
a CO₂ shift unit, being an electrically heated Reverse Water Gas Shift (e-RWGS) unit, and being arranged to receive the first feed and the second feed and provide a first synthesis gas stream,
a water removal unit arranged to remove water from said first synthesis gas stream and provide a second synthesis gas stream,
a methanol synthesis section, arranged to receive at least a portion of said second synthesis gas stream, and—optionally—a portion of said second feed and/or said first feed, and provide a product stream comprising methanol and an off-gas stream.

A process for producing a product stream comprising methanol, in a methanol plant described herein is also provided. The process comprising the steps of:
providing a methanol plant as described herein,
supplying first feed and second feed to the CO₂ shift unit, and providing a first synthesis gas stream,
supplying the first synthesis gas stream to the water removal unit and removing water from said first synthesis gas stream so as to provide a second synthesis gas stream,
supplying at least a portion of said second synthesis gas stream, and—optionally—a portion of said second feed and/or said first feed, to the methanol synthesis section, and providing a product stream comprising methanol and an off-gas stream.

A process is also provided for retrofitting a carbon dioxide-to-methanol plant (X), in which said plant (X) comprises:
a first feed comprising CO₂ to said plant, and a second feed comprising H₂ to said plant;
or a combined feed comprising CO₂ and H₂ to said plant;
a methanol synthesis section.

The retrofitting process comprises the steps of:
incorporating a CO₂ shift unit, being an electrically heated Reverse Water Gas Shift (e-RWGS) unit, and a water removal unit in said plant, upstream said methanol synthesis section,
arranging at least a portion of said first feed and said second feed, or of said combined feed comprising CO₂ and H₂, to be supplied to the CO₂ shift unit so as to provide a first synthesis gas stream,
feeding at least a portion of said first synthesis gas stream from the CO₂ shift unit to the water removal unit, to remove water from said first synthesis gas stream and provide a second synthesis gas stream,
arranging at least a portion of the second synthesis gas stream to be supplied to a compressor and compressing it, so as to provide a compressed second synthesis gas stream,
arranging at least a portion of the compressed second synthesis gas stream to be supplied to the methanol synthesis section, and providing a product stream comprising methanol.

The present invention allows for an increase in the methanol production in an existing CO₂-to-MeOH plant by shifting the feed gas to a more reactive gas mixture in an upstream CO₂ shift unit. By means of the present technology, all central equipment of the existing CO₂-to-MeOH plant can be reused as the concept allows for replacing the existing gas flow with a new and more reactive gas flow to the methanol synthesis section.

Further details of the technology are provided in the enclosed dependent claims, figures, and examples.

### LEGENDS

The technology is illustrated by means of the following schematic illustrations, in which:

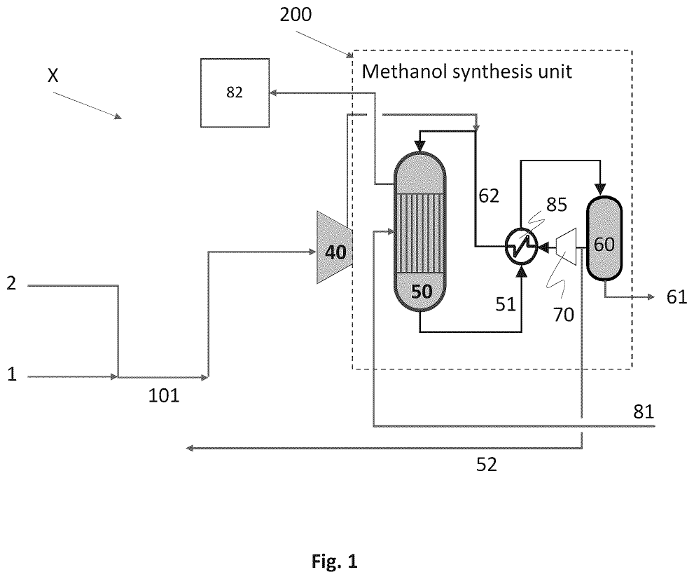

FIG. 1 shows a standard layout for a conventional CO₂-to-MeOH plant.

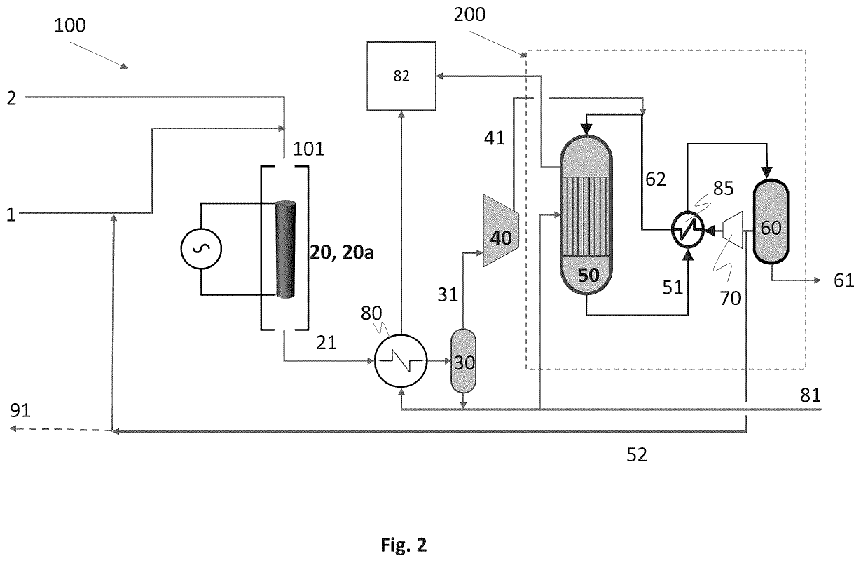

FIGS. 2-4 show various layouts for a CO₂-to-MeOH plant according to the invention.

### DETAILED DISCLOSURE

Unless otherwise specified, any given percentages for gas content are % by volume. All feeds are preheated as required.

The term “synthesis gas” (abbreviated to “syngas”) is meant to denote a gas comprising hydrogen, carbon monoxide, carbon dioxide and small amounts of other gasses, such as argon, nitrogen, methane, etc.

In the “classical” configuration of a CO₂-to-MeOH plant, CO₂ and H₂ are compressed as is and fed to a methanol synthesis section. Consequently, the methanol synthesis reactor in the methanol synthesis section has to accommodate the following two reactions:

CO₂+H₂<=>CO+H₂O

CO+2H₂<=>CH₃OH

This requires a relatively large catalyst volume. Additionally, the high byproduct of water leads to high degree of sintering of a classical methanol synthesis catalyst.

The present invention describes a process for upgrading (i.e. retro-fitting) a conventional CO₂-to-MeOH plant, based on a conventional plant comprising a compressor and a methanol synthesis section. The methanol production capacity can be increased by installing a CO₂ shift unit on the CO₂ feed line to convert a substantial part of the CO₂ to CO. This is done by using the hydrogen stream and reacting this with the CO₂ to perform the reverse water gas shift reaction according to:

CO₂+H₂<=>CO+H₂O

and subsequently removing the principal part of the water in a water removal unit.

By introducing the CO₂ shift unit and the water removal unit several advantages are obtained. Firstly, the number of gas molecules going into the methanol synthesis section is reduced according to the stoichiometry:

CO₂(g)+3H₂(g)-(CO₂shift unit)->

xCO(g)+(1−x)CO₂(g)+(3−x)H₂(g)+xH₂O(g)-(cooling)->

xCO(g)+(1−x)CO₂(g)+(3−x)H₂(g)+xH₂O(I)-(water removal unit)->

xCO(g)+(1−x)CO₂(g)+(3−x)H₂(g)-(methanol synthesis section)->

CH₃OH(g)+(1−x)H₂O(g)

Accordingly, the amount of gas molecules in the methanol synthesis is reduced by x, where x is the conversion degree in the CO₂ shift unit. The reduced number of molecules allows for increased capacity in compression stages, methanol synthesis sections, as well as other equipment, and the total feed to the plant can be increased accordingly.

In addition, by shifting the CO₂ to CO, an increased activity of the methanol synthesis is achieved, as the reaction rate for methanol production is faster from CO than CO₂ due to both kinetics aspects of the catalyst but also because the byproduct of water is reduced in the reactor, which has a deactivating effect on the traditional methanol synthesis catalyst due to especially sintering.

As an added feature, the invention allows for reducing the amount of water in the methanol product. This also means that removal of water in a prospective downstream distillation section is reduced, which similarly leaves room for additional methanol production capacity in the existing equipment of the CO₂-to-MeOH plant.

Accordingly, in a first embodiment, a methanol plant is provided. The methanol plant comprises:
a first feed comprising CO₂ to said plant,
a second feed comprising H₂ to said plant,
a CO₂ shift unit, being an electrically heated Reverse Water Gas Shift (e-RWGS) unit,
a water removal unit, and
a methanol synthesis section.

A first feed comprising carbon dioxide is provided to the plant. The first feed suitably comprises more than 90% CO₂, preferably more than 95% CO₂, preferably more than 99% CO₂. The first feed may in addition to CO₂ comprise minor amounts of, for example, steam, oxygen, nitrogen, oxygenates, amines, ammonia, carbon monoxide, and/or hydrocarbons. The first feed suitably comprises only low amounts of hydrocarbon, such as for example less than 5% hydrocarbons or less than 3% hydrocarbons or less than 1% hydrocarbons.

In an embodiment a higher fraction of hydrocarbon can be present in said first feed, such as up to 10% or up to 20%. For high content of hydrocarbons said stream suitably should also contain some steam, which should suitably be added in a ratio of 1-3 relative to the content of hydrocarbons.

A second feed comprising hydrogen is provided to the plant. Suitably, the second feed consists essentially of hydrogen. The second feed is suitably “hydrogen rich” meaning that the major portion of this feed is hydrogen, i.e. over 75%, such as over 85%, preferably over 90%, more preferably over 95%, even more preferably over 99% of this feed is hydrogen. One source of the second feed can be one or more electrolyser units. In addition to hydrogen the second feed may for example comprise steam, nitrogen, argon, carbon monoxide, carbon dioxide, and/or hydrocarbons. In some cases, a minor content of oxygen may be present in this second feed, typically less than 100 ppm. The second feed suitably comprises only low amounts of hydrocarbon, such as for example less than 5% hydrocarbons or less than 3% hydrocarbons or less than 1% hydrocarbons.

In one aspect, the first feed and the second feed are combined prior to being fed to the CO₂ shift unit.

The CO₂ shift unit is arranged to receive the first feed and the second feed and provide a first synthesis gas stream. The shift unit is an electrically heated Reverse Water Gas Shift (e-RWGS) unit.

Overall, the configuration of the invention allows for facilitating the reverse water gas shift reaction and the methanation reaction within an e-RWGS unit without having a side reaction of carbon formation on neither the catalyst nor metallic surfaces, as the methanation reaction counterintuitively mitigates this. The specific configuration of the e-RWGS unit which allows for increasing the temperature from a relative low inlet temperature to a very high product gas temperature of more than 500° C., preferably more than 800° C., and even more preferably more than 900° C. or 1000° C. means that the resulting methane formed from the methanation reaction will occur in the first part of the unit, but when exceeding ca. 600-800° C. this methane will start to be converted by the reverse methanation reaction back to a product of CO₂ and H₂. This configuration elegantly allows for removing some of the CO and generation of some H₂O inside the catalyst bed in the temperature region where CO reduction is a problem, but then allows for reproducing the CO in the high temperature zone with low or no carbon potential. Effectively, utilizing the high product gas temperature then means that the final product can be delivered with a very low methane concentration, despite the methane has a peak concentration somewhere along the reaction zone. In an embodiment, the e-RWGS unit is operated with none, or very little, methane in the feed and only very little methane in the product gas, but with a peak in methane concentration inside the reaction zone higher than in the feed and/or product gas. In some cases, this peak methane concentration inside the reaction zone may be an order of magnitude higher than the inlet and outlet methane concentrations.

In a particular embodiment, the e-RWGS unit is operated with a gas exit temperature of more than 500° C., preferably more than 800° C., more preferably more than 900° C. and most preferably more than 1000° C.

In an embodiment of the invention where the e-RWGS unit is active for methane conversion to CO, the overall yield of synthesis gas is positively increased when the carbon feedstock contains hydrocarbons because these are converted to syngas in the reactor. In a configuration without a e-RWGS unit capable of converting hydrocarbons to synthesis gas, such hydrocarbons would end up as inert gas in the methanol loop. In an embodiment of the e-RWGS unit of the invention, the structured catalyst has a first reaction zone disposed closest to the first end of said structured catalyst, wherein the first reaction zone has an overall exothermic reaction, and a second reaction zone disposed closest to the second end of said structured catalyst, wherein the second reaction zone has an overall endothermic reaction. Preferably, said first reaction zone has an extension of from 5% to 60% of the length of the structured catalyst from its first to its second end. Here, the reaction zone means the volume of the reactor system catalyzing the methanation and reverse water gas shift reactions as evaluated along the flowpath through the catalytic area.

The combined activity for both reverse water gas shift and methanation in an e-RWGS unit entails that the reaction scheme inside the reactor will start out as exothermic in the first part of the reactor system but end as endothermic towards the exit of the reactor system. This relates to the heat of reaction (Qᵣ) added or removed during the reaction, according to the general heat balance of the plug flow reactor system:

$$F \cdot {C}_{pm} \cdot dT / dV = {Q}_{add} + {Q}_{r} = {Q}_{add} + \sum ( - {Δ}_{r} {H}_{i} ) \cdot ( - {r}_{i} )$$

where F is the flow rate of process gas, Cpm is the heat capacity, V the volume of the reaction zone, T the temperature, Q_{add} the energy supply/removal from the surrounding, and Qᵣ the energy supply/removal associated with chemical reactions which are given as the sum of all chemical reactions facilitated within the volume and calculated as the product between the reaction enthalpy and the rate of reaction of a given reaction. In an embodiment of the reactor system of the invention, the temperature of the feedstock at the inlet of the pressure shell is between 200° C. and 500° C., preferably between 200° C. and 400° C.

In an embodiment of the e-RWGS unit, the concentration of methane is higher in the partly catalyzed feedstock inside at least a part of the structured catalyst than in the feedstock and in the first product gas.

In an embodiment of the e-RWGS unit, the temperature of the structured catalyst is continuously increasing from the first end to the second end of the structured catalyst.

In another preferred embodiment, hydrogen generation and the CO₂ shift unit are integrated and comprise a Solid Oxide Electrolysis Cell (SOEC), and an eRWGS unit. Details of this unit are as per co-pending publication PCT/EP2021/086678.

A water removal unit is arranged to remove water from the first synthesis gas stream (from the CO₂ shift unit) and provide a second synthesis gas stream. At the same time, a water-rich stream is provided. The second synthesis gas stream has a lower water content than the first synthesis gas stream and will typically be below 5%, preferably below 1%. The water removal unit is suitably a flash separation unit.

The methanol synthesis section is arranged to receive at least a portion of said second synthesis gas stream, and provide a product stream comprising methanol and an off-gas stream. Depending on the composition of the second synthesis gas stream, a portion of the second feed and/or the first feed may optionally be fed to the methanol synthesis section, together with the second synthesis gas stream. The product stream comprising methanol may be further purified to the required commercial grade, downstream the methanol synthesis section.

As illustrated in FIGS. 1 and 2, the methanol synthesis section (also called a “methanol loop”) comprises methanol synthesis reactor and a methanol purification unit. Synthesis gas stream enters the methanol synthesis reactor, where it is converted in the presence of a catalyst to a raw product stream comprising methanol. The raw product stream comprising methanol may be cooled in a heat exchanger before being fed to a methanol purification unit. The methanol purification unit provides a product stream comprising methanol and a recycle stream, a part of which is purged from the methanol synthesis section as an off-gas stream. Other arrangements of the internal components of the methanol synthesis section may be known to the skilled person.

In one aspect, a compressor is located between the water removal unit and the methanol synthesis section and arranged to compress the second synthesis gas stream. A compressed second synthesis gas stream is thus provided to the methanol synthesis section. This arrangement allows efficient use of the compressor which is present in the carbon dioxide-to-methanol plant prior to refitting, and also allows the second synthesis gas stream to reach the required pressure upstream the methanol synthesis section.

In one aspect, at least a portion of the off-gas stream from the methanol synthesis section is arranged to be fed to the inlet of the CO₂ shift unit, preferably in admixture with the first stream comprising CO₂. In this manner, carbon utilisation in the plant can be increased.

In one aspect of the invention, the methanol plant further comprises a feed compressor. The feed compressor is arranged to receive the first feed, the second feed or the combined feed and to output a compressed stream, upstream the CO₂ shift unit.

The methanol plant may further comprise a first heat exchanger, arranged to cool the first synthesis gas stream from the CO₂ shift unit to provide a cooled first synthesis gas stream.

The methanol plant may further comprise a feed-effluent heat exchanger, arranged to receive the compressed stream from the feed compressor and heat it to a heated stream in heat exchange with the first synthesis gas stream or the cooled first synthesis gas stream, which is cooled to form a further cooled first synthesis gas stream.

The methanol plant may further comprise an additional heat exchanger, preferably a synthesis gas distillation column, arranged downstream the feed-effluent heat exchanger and arranged to cool the further cooled first synthesis gas stream.

The methanol plant may also further comprise a hydrogen recovery unit arranged to receive at least a portion of the off-gas stream from the methanol synthesis section and to output a hydrogen rich gas stream and a reject stream. Preferably at least a portion of the hydrogen rich gas stream is arranged to be recycled upstream the CO₂ shift unit and/or wherein at least a portion of said hydrogen rich gas stream is arranged to be recycled upstream the methanol synthesis section.

The methanol plant suitably further comprises a low pressure separator arranged to receive at least a portion of the product stream comprising methanol from the methanol synthesis section and to output a purified methanol stream and a carbon-rich stream, preferably wherein at least a portion of said carbon-rich off-gas stream is arranged to be recycled upstream the CO₂ shift unit and/or wherein at least a portion of said carbon-rich stream is arranged to be recycled upstream the methanol synthesis section.

The methanol plant may further comprise a methanol distillation section arranged to receive at least a portion of the product stream comprising methanol from the methanol synthesis section or at least a portion of the purified methanol stream from the low pressure separator and to output a high-purity methanol stream and a higher alcohols stream, preferably wherein at least a portion of said higher alcohols stream is arranged to be recycled upstream the CO₂ shift unit. The “higher alcohols stream” is essentially a water-methanol mixture (more or less equal amounts) with higher alcohols (ethanol, propanol etc), the higher alcohols typically being around 10 weight %.

In this aspect of the methanol plant, at least a portion of the heat required for the methanol distillation section may be arranged to be provided from the first heat exchanger.

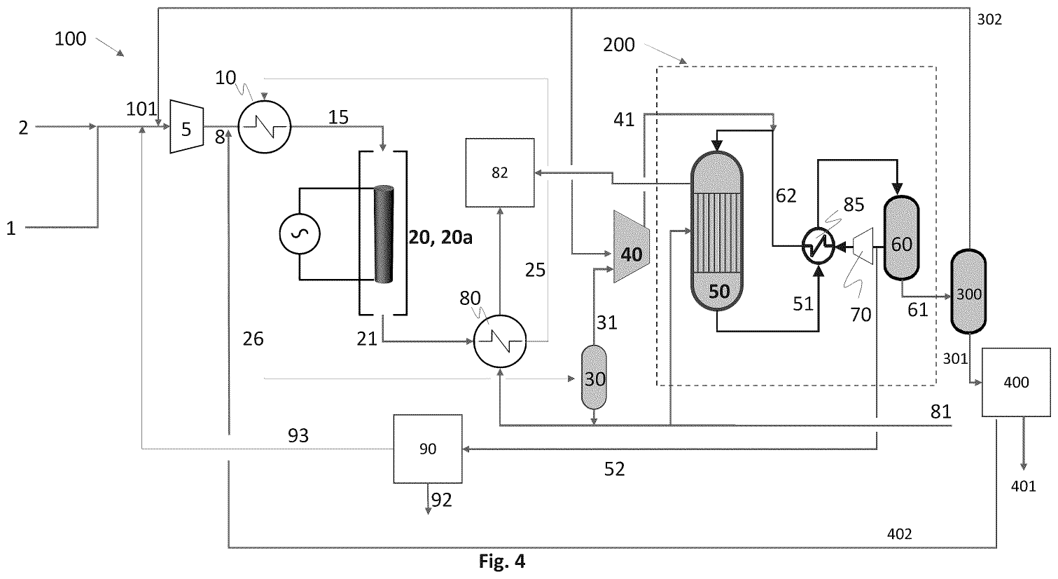

The methanol plant of the invention may further comprise a hydrogen recovery unit arranged to receive at least a portion of the off-gas stream and to output a hydrogen rich gas stream and a reject stream, preferably wherein at least a portion of said hydrogen rich gas stream is arranged to be mixed with said first feed, said second feed or said combined feed upstream the CO₂ shift unit. Although this is illustrated in connection with FIG. 4, this aspect is relevant for all embodiments of the invention. The hydrogen recovery unit can be of a pressure swing adsorption (PSA) type or a membrane type.

Using a hydrogen recovery unit as described above can have the following different effects:
reduce the requirement of H₂ feedstock and maintain methanol production at the same level when using a PSA type hydrogen recovery unit.
reduce the requirement of H₂ and CO₂ feedstocks and maintain methanol production at the same level when using a membrane type hydrogen recovery unit.
increase methanol production while maintaining CO₂ feedstock and H₂ feedstock at the same level when using a PSA type hydrogen recovery unit.
increase methanol production even further while maintaining CO₂ feedstock and H₂ feedstock at the same level when using a membrane type hydrogen recovery unit.

The present technology also provides a process for producing a product stream comprising methanol, in a methanol plant as described herein. The process comprises the steps of:
providing a methanol plant as described above,
supplying first feed and second feed to the CO₂ shift unit, being an electrically heated Reverse Water Gas Shift (e-RWGS) unit, and providing a first synthesis gas stream,
supplying the first synthesis gas stream to the water removal unit, and removing water from said first synthesis gas stream so as to provide a second synthesis gas stream,
supplying at least a portion of said second synthesis gas stream, and—optionally—a portion of said second feed and/or said first feed, to the methanol synthesis section, and providing a product stream comprising methanol and an off-gas stream.

All details of the methanol plant described herein are also relevant to the process described herein, mutatis mutandis. In particular, the process may involve a step of combining the first feed and the second feed to a combined feed prior to feeding this to the CO₂ shift unit.

In one aspect of this process, the first feed may additionally comprise up to 20%, such as up to 10% hydrocarbons. Also, in this aspect, the first feed may additionally comprise steam, preferably wherein the steam is present in a ratio of 1-3 relative to the content of hydrocarbons.

A process is also provided for retrofitting a (conventional) carbon dioxide-to-methanol plant, which plant comprises:
a first feed comprising CO₂ to said plant, and a second feed comprising H₂ to said plant;
or a combined feed comprising CO₂ and H₂ to said plant;
a methanol synthesis section;

The retrofitting process comprises the steps of:
incorporating a CO₂ shift unit, being an electrically heated Reverse Water Gas Shift (e-RWGS) unit, and a water removal unit in said plant, upstream said methanol synthesis section,
arranging at least a portion of said first feed and said second feed, or of said combined feed comprising CO₂ and H₂, to be supplied to the CO₂ shift unit so as to provide a first synthesis gas stream,
feeding at least a portion of said first synthesis gas stream from the CO₂ shift unit to the water removal unit, to remove water from said first synthesis gas stream and provide a second synthesis gas stream,
arranging at least a portion of the second synthesis gas stream to be supplied to a compressor and compressing it, so as to provide a compressed second synthesis gas stream,
arranging at least a portion of the compressed second synthesis gas stream to be supplied to the methanol synthesis section, and providing a product stream comprising methanol in said methanol synthesis section.

A conventional carbon dioxide-to-methanol plant is shown in FIG. 1, prior to retrofitting. FIG. 2 shows the retrofitted plant. All details relating to the methanol plant, described above, are also relevant for the refitting process.

Retrofitting is designed to make best use of the existing components of the conventional carbon dioxide-to-methanol plant. Therefore, in the case where the carbon dioxide-to-methanol plant comprises a compressor, and—before the retrofitting process-said compressor is arranged to compress the combined feed-after said retrofitting process—the compressor is suitably arranged to compress at least a portion of the second synthesis gas stream and provide a compressed second synthesis gas stream.

In one aspect, the combined feed also comprises hydrocarbons. In a further aspect, the combined stream is obtained by mixing a biogas with hydrogen.

The retrofitting process improves methanol production capacity in the carbon dioxide-to-methanol plant. Suitably, the methanol synthesis section of said carbon dioxide-to-methanol plant (X) remains unchanged under said retrofitting process. In other words, the methanol synthesis section of the conventional carbon dioxide-to-methanol plant is mechanically identical to the methanol synthesis section of the retrofitted carbon dioxide-to-methanol plant. By this is understood that the central equipment of the methanol synthesis section is unchanged. The “methanol synthesis section” refers to the section which converts a syngas stream to a raw methanol stream, excluding any product refinement sections, such as distillation sections, but not excluding flash separation section.

In this aspect, the methanol synthesis reactor will maintain the same dimensions, the recycle compressor will be of the same with the same nameplate capacity, the flash separation vessel will be the same, with the same dimensions, and the piping will be the same, with the same dimensions, to name some examples of unchanged equipment.

In this particular aspect, where little or no modification to the methanol synthesis section is carried out, the production of methanol from the carbon dioxide-to-methanol plant after the retrofitting process is at least 10% higher, 20% higher, 33% higher, 50% higher, 100% higher or 250% higher than the methanol production of the conventional carbon dioxide-to-methanol plant (X) prior to the retrofitting process when evaluated on the flow of methanol molecules from the methanol synthesis section.

Although it is of practical interest to keep the methanol synthesis section substantially unchanged during retrofitting process, further improvements in methanol production capacity can be achieved by upgrading various components of the methanol synthesis section. In the case, therefore, when the methanol synthesis section comprises a recycle compressor, a heat exchanger, a flash separation vessel, and/or a cooling unit, the refitting process may comprise one or more further steps of upgrading one or more of said recycle compressor, heat exchanger, flash separation vessel and cooling unit. In that a component is “upgraded” is meant that its capacity is increased by exchanging said component with a new one with larger capacity, making a similar component in parallel to the one already installed to share the capacity with the first, and/or rebuilding said component but still reusing parts of the existing component.

An “upgraded” recycle compressor has an increase in capacity from 10% to 200%, preferably from 10% to 100%, in terms of the electrical duty required to drive the compressor, compared to the compressor before upgrading.

An “upgraded” heat exchanger and/or cooling unit has an increase in capacity from 10% to 200%, preferably from 10% to 100%, in terms of the transfer duty, compared to the heat exchanger/cooling unit before upgrading.

An “upgraded” flash separation vessel and/or piping has an increase in capacity from 10% to 200%, preferably from 10% to 100%, in terms of the volume flow, compared to the flash separation vessel and/or piping before upgrading.

In one particular aspect, in which the methanol synthesis section comprises a methanol synthesis reactor, the refitting process may comprise one or more further steps of reducing the catalyst loading in the methanol synthesis reactor. The reactor is thus “short loaded” (with less catalyst) to avoid too high pressure drop in the reactor. This may seem counterintuitive for increasing methanol production capacity, but because the reactivity of a synthesis gas is increased substantially when converting CO₂ to CO and removing water upstream the reactor, more methanol can be produced from a smaller catalyst volume, due to increased reactivity and a larger thermodynamic driving force towards the methanol product.

In the case where one or more units of the methanol synthesis section is upgraded to increase the processing capacity thereof, and/or where the catalyst loading in the methanol synthesis reactor is reduced, the production of methanol from the carbon dioxide-to-methanol plant after the retrofitting process is at least 10% higher, 20% higher, 33% higher, 50% higher, 100% higher, 250% higher, 500% higher, 750% higher or 1000% higher than the methanol production of the conventional carbon dioxide-to-methanol plant (X) prior to the retrofitting process when evaluated on the flow of methanol molecules from the methanol synthesis section.

When retrofitting said process for increased production capacity, an increased capacity of hydrogen and carbon containing feedstocks are assumed available.

In an embodiment, said feedstock expansion is obtained by securing a large feedstock of CO₂. The boost in capacity can be obtained by by utilizing the CH₄ fraction of natural gas or biogas in addition to the CO₂ fraction. This can happen as the catalyst in the shift unit is steam reforming active and can therefore react CH₄ with H₂O and CO₂ to produce CO and H₂.

In another embodiment said feedstock expansion is obtained by securing a larger feedstock of H₂ by adding (increased) water electrolysis capacity. In another embodiment, said feedstock expansion is achieved by importing a hydrocarbon containing gas, said hydrocarbon containing gas may be natural gas, or preferentially biogas.

The retrofitting process also applies where the first plant is constructed to make MeOH from a CO₂ fraction from a biogas plant, and where it is desired at a later stage to make MeOH from the total biogas (also containing the CH₄ as well as the CO₂).

The current invention therefore describes a method where an existing CO₂-to-MeOH plant is retrofitted by installing a CO₂ shift unit being an electrically heated Reverse Water Gas Shift (e-RWGS) unit, up-stream the methanol synthesis section. This involves increasing the hydrogen and CO₂ feed ratio to the plant to effectively have a larger reactant flow into the plant. In a preferred embodiment, hydrogen generation and the CO₂ shift unit are integrated and comprise a Solid Oxide Electrolysis Cell (SOEC), and an eRWGS unit. In this embodiment, a feedstock of steam is heated and sent to the SOEC which at least partially converts the steam into H₂. The hot effluent of the SOEC is directly mixed with at least a portion of the CO₂, and potentially all or a portion of the existing H₂ feedstock, and potentially any carbon containing by-products from the plant (such as alcohol by-products from the distillation section) and fed directly to the eRWGS to facilitate at least partial conversion of the CO₂ to CO. Subsequently, the water of the resulting synthesis gas is removed in a water removal unit to in this way reduce the feed flow of the gas. In an embodiment the flow of the new synthesis gas is substantially equal to the previous feed flow of the original H₂ and CO₂ feedstock.

In another, but similar embodiment, the increased H₂ capacity is achieved by using alkaline electrolysis (or PEM), while still using the eRWGS configuration.

In a particular aspect of the retrofitting process, this may further comprise a step of arranging a hydrogen recovery unit so as to receive at least a portion of the off-gas stream from the methanol synthesis section and to output a hydrogen rich gas stream and a reject stream, preferably wherein at least a portion of said hydrogen rich gas stream is arranged to be recycled upstream the CO₂ shift unit and/or wherein at least a portion of said hydrogen rich gas stream is arranged to be recycled upstream the compressor.

In a further aspect, the retrofitting process may further comprise a step of arranging a hydrogen recovery unit so as to receive at least a portion of the off-gas stream from the methanol synthesis section and to output a hydrogen rich gas stream and a reject stream, preferably wherein at least a portion of said hydrogen rich gas stream is arranged to be recycled upstream the CO₂ shift unit and/or wherein at least a portion of said hydrogen rich gas stream is arranged to be recycled upstream the methanol synthesis section.

### Specific Embodiments

FIG. 1 shows a conventional carbon dioxide-to-methanol plant (X), comprising:
a combined feed 101 comprising CO₂ 1 and H₂ 2,
a methanol synthesis section 200 (methanol loop).

The combined feed 101 is compressed in compressor 40 before being fed to methanol synthesis section 200. In the illustrated embodiment, the methanol synthesis section 200 comprises a methanol synthesis reactor 50 and a methanol purification unit 60.

The combined feed 101 is converted into a raw product stream 51 comprising methanol in the methanol synthesis reactor 50. The raw product stream 51 comprising methanol is cooled in second heat exchanger 85 before being fed to methanol purification unit 60. Methanol purification unit 60 provides a product stream comprising methanol 61 and a recycle stream 62. A part of the recycle stream 62 is purged from the loop as an off-gas stream 52. The recycle stream 62 is warmed in the other side of the second heat exchanger 85, and fed to the inlet of the methanol synthesis section 200, preferably in admixture with the combined feed 101. A compressor 70 may be arranged to compress the recycle stream 62, prior to the second heat exchanger 85. As shown, part of the recycle stream 62 is purged from the loop as an off-gas stream 52

Methanol synthesis reactor 50 can be cooled by a water feed 81, and outputs process steam 82.

FIG. 2 shows a carbon dioxide-to-methanol plant, retro-fitted (re-fitted) according to the invention. As can be seen, many components of the plant remain unchanged during refitting, and are indicated with the same reference numbers. The combined feed 101 comprising CO₂ and H₂ to the plant also remains the same. Compared to FIG. 1, the layout of FIG. 2 includes a CO₂ shift unit 20 and a water removal unit 30, upstream said methanol synthesis section 200. In the illustrated embodiment, the CO₂ shift unit 20 is an electrically heated Reverse Water Gas Shift (e-RWGS) unit 20a.

The combined feed 101 comprising CO₂ and H₂ is supplied to the CO₂ shift unit 20, where it is converted into a first synthesis gas stream 21. The first synthesis gas stream 21 is first passed through first heat exchanger 80 where it is cooled, preferably using a water feed 81.

The first synthesis gas stream 21 is then fed from the CO₂ shift unit 20 to the water removal unit 30, where water is removed to provide a second synthesis gas stream 31. Water removed at this point may be combined with the general water feed 81 in the plant.

The second synthesis gas stream 31 is then supplied to a compressor 40 where it is compressed, so as to provide a compressed second synthesis gas stream 41. Compressed second synthesis gas stream 41 is fed to the methanol synthesis section 200, where a product stream 61 comprising methanol and off-gas stream 52 are produced. Remaining components and streams in FIG. 2 are as in FIG. 1. A portion of the off-gas stream 52 may be supplied to another process as fuel stream 91.

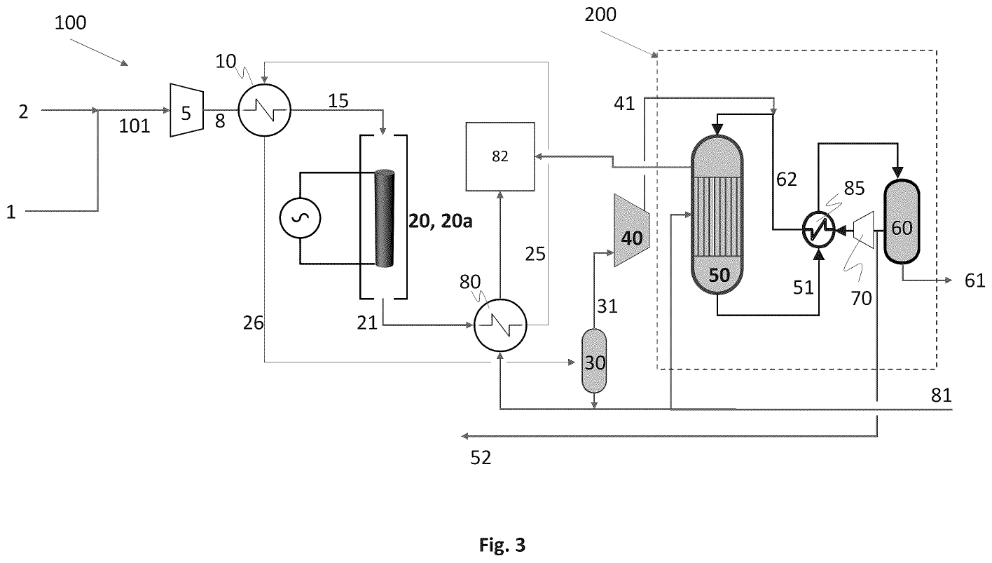

A further embodiment of the invention is illustrated in FIG. 3. The first feed (1) and second feed (2) are combined to a combined feed (101) prior to being compressed in a feed compressor (5). The compressed stream (8) is heated, for instance to 140° C., in a feed-effluent heat exchanger (10) producing heated stream (15) which enters the CO₂ shift unit (20).

Optionally, it may not be necessary to compress the combined feed (101) in a compressor (5) depending on the pressures of first feed (1) and second feed (2). Optionally, only one of first feed (1) and second feed (2) needs compression in a compressor (5) before entering the feed-effluent heat exchanger (10) together with the other feed.

The hot first synthesis gas stream (21) from the CO₂ shift unit (20) is utilized to produce steam in a first heat exchanger (i.e. waste heat boiler 80).

The now cooled first synthesis gas stream (25) from the waste heat boiler (80) is used to preheat the compressed stream (8) in said feed-effluent heat exchanger (10). Thereby the stream (25) is further cooled to stream (26) before entering the water removal unit (30).

Optionally, the heat in the stream (26) can be used as heating duty, for instance, in a distillation column reboiler in an associated distillation section of the methanol plant (reboiler and distillation section not shown), before entering the water removal unit (30).

Remaining components in FIG. 3 are as in FIG. 2. In the water removal unit (30) water is removed to produce the second synthesis gas stream (31). Said synthesis gas stream (31) is compressed in compressor (40) to provide a compressed second synthesis gas stream (41) to the methanol synthesis section (200).

The layout in FIG. 4 is based on that of FIG. 3. As shown in FIG. 4, a hydrogen recovery unit (90) can be included on the off-gas stream (52) containing H₂, CO and CO₂ to produce a H₂ rich stream (93) containing limited amounts of CO and CO₂. This hydrogen rich stream (93) can be added to the first feed (1) or second feed (2) or combined feed (101) thereby limiting the required amount of said second feed (2), optionally also of first feed (1) depending of type of hydrogen recovery unit, and still producing an unchanged amount of methanol. This can be beneficial if the second feed (2) is produced in an electrolyser unit which requires a lot of electricity (not shown).

Said hydrogen recovery unit (90) can be of a pressure swing adsorption (PSA) type or a membrane type but is not limited to said types. The PSA type would produce a H₂ rich gas stream of essentially pure H₂ and an a reject stream (92). A membrane type would produce a H₂ rich gas containing primarily H₂, secondarily CO and CO₂, and a reject stream (92). The layout of FIG. 4 further comprises a a low pressure separator (300) arranged to receive at least a portion of the product stream (61) comprising methanol from the methanol synthesis section (200) and to output a purified methanol stream (301) and a carbon-rich stream (302), wherein at least a portion of said carbon-rich off-gas stream (302) is arranged to be recycled upstream the feed compressor (5).

The layout of FIG. 4 further comprises a methanol distillation section (400) arranged to receive at least a portion of the purified methanol stream (301) from the low pressure separator (300) and to output a high-purity methanol stream (401) and a higher alcohols stream (402), wherein at least a portion of said higher alcohols stream (402) is arranged to be recycled upstream the feed-effluent heat exchanger (10).

### COMPARATIVE EXAMPLE

In a comparative example a combined feed of 0.2% C₂H₆, 0.4% CH₄, 3.2% CO, 20.6% CO₂, 75.4% H₂, and 0.3% N₂ at a feed rate of 8282 Nm³/h is fed to a methanol synthesis section. Using a boiling water type methanol reactor with inlet temperature of 237° C., an inlet pressure of 90 barg and outlet temperature of 264° C. and an internal recycle so the methanol reactor feed flow is 52254 Nm³/h and the recycle compressor duty is 128 kW at 81% efficiency a methanol product of 2610 kg/h is produced. This is the raw methanol product with an actual methanol concentration of 63 wt % in the mixed liquid coming from the separation vessel before prospective methanol distillation.

### Example 1

In a first example of the invention a combined feed of 0.2% C₂H₆, 0.4% CH₄, 3.2% CO, 20.6% CO₂, 75.4% H₂, and 0.3% N₂ at a feed rate of 13856 Nm³/h is fed to an e-RWGS section where the gas is shifted and water removed to produce a feed of 0.2% CH₄, 26.5% CO, 5.2% CO₂, 68.0% H₂, and 0.2% N₂ at a flow of 10685 Nm³/h fed to a methanol section. The drop in flow over the e-RWGS section is from condensation and thereby removal of water. This feed is compressed to 90 barg and fed to the methanol section. Using a boiling water type methanol reactor with inlet temperature of 240° C. and outlet temperature of 265° C. (the methanol reactor in this case is the exact same dimensions as the methanol reactor in the comparative example) and an internal recycle so the methanol reactor feed flow is 29615 Nm³/h and the recycle compressor duty is 41 kW at 81% efficiency a methanol product of 4575 kg/h is produced. This is the raw methanol product with an actual methanol concentration of 91 wt % in the mixed liquid coming from the separation vessel before prospective methanol distillation. Notice that this is much higher than the comparative example due to a much lower water content in the feed entering the methanol synthesis loop.

In this case the methanol reactor only receives a flow of 56% relative to the comparative example, the recycle compressor only has a duty of 32% relative to the comparative example, which illustrates that no modification would be needed if the comparative example was revamped into example 1. Nevertheless, the methanol production capacity in this Example is boosted by +75%.

### Example 2

In a second example of the invention a combined feed of 0.2% C₂H₆, 0.4% CH₄, 3.2% CO, 20.6% CO₂, 75.4% H₂, and 0.3% N₂ at a feed rate of 32280 Nm³/h is fed to an e-RWGS section where the gas is shifted and water removed to produce a feed of 0.2% CH_{4,} 26.5% CO, 5.2% CO₂, 68.0% H₂, and 0.2% N₂ at a flow of 24893 Nm³/h fed to a methanol section. The drop in flow over the e-RWGS section is from condensation and thereby removal of water. This feed is compressed to 90 barg and fed to the methanol section. Using a boiling water type methanol reactor with inlet temperature of 240° C. and outlet temperature of 274° C. (the methanol reactor in this case is the exact same dimensions as the methanol reactor in the comparative example) and an internal recycle so the methanol reactor feed flow is 95548 Nm³/h and the recycle compressor duty is 289 KW at 81% efficiency a methanol product of 10425 kg/h is produced. This is the raw methanol product with an actual methanol concentration of 91 wt % in the mixed liquid coming from the separation vessel before prospective methanol distillation.

In this case the recycle compressor only has a duty of 195% % relative to the comparative example, which illustrates that some modification to the loop might be necessary in this case. However, the methanol reactor, despite a higher flow could possibly be the same. Overall, this approach boosts production by +299% relative to the comparative example in a comparative methanol loop. The performance of example 2 can be derived with a methanol loop from the comparative example with some minor modification to equipment such as the recycle compressor.

The present invention has been described with reference to a number of embodiments and figures. However, the skilled person is able to select and combine various embodiments within the scope of the invention, which is defined by the appended claims. All documents referenced herein are incorporated by reference.

The invention claimed is:

1. A methanol plant, said methanol plant comprising:
a first feed comprising CO2 to said plant,
a second feed comprising H₂ to said plant,
a CO2 shift unit, being an electrically heated Reverse Water Gas Shift unit, and being arranged to receive the first feed and the second feed and provide a first synthesis gas stream,
a water removal unit arranged to remove water from said first synthesis gas stream and provide a second synthesis gas stream,
a methanol synthesis section, arranged to receive at least a portion of said second synthesis gas stream, and—optionally—a portion of said second feed and/or said first feed, and provide a product stream comprising methanol and an off-gas stream.

2. The methanol plant according to claim 1, wherein a compressor is located between the water removal unit and the methanol synthesis section, and arranged to compress said second synthesis gas stream and provide a compressed second synthesis gas stream to the methanol synthesis section.

3. The methanol plant according to claim 1, wherein the first feed and the second feed are combined to a combined feed prior to being fed to the CO2 shift unit.

4. The methanol plant according to claim 1, wherein the water removal unit is a flash separation unit.

5. The methanol plant according to claim 1, wherein at least a portion of the off-gas stream from the methanol synthesis section is arranged to be fed to the inlet of the CO2 shift unit.

6. The methanol plant according to claim 3, further comprising a feed compressor, wherein the feed compressor is arranged to receive the first feed, the second feed or the combined feed and to output a compressed stream, upstream the CO2 shift unit.

7. The methanol plant according to claim 1, further comprising a first heat exchanger, arranged to cool the first synthesis gas stream from the CO2 shift unit to provide a cooled first synthesis gas stream.

8. The methanol plant according to any one of the preceding claim 1, further comprising a feed-effluent heat exchanger, arranged to receive the compressed stream from the feed compressor and heat it to a heated stream in heat exchange with the first synthesis gas stream or the cooled first synthesis gas stream, which is cooled to form a further cooled first synthesis gas stream.

9. The methanol plant according to claim 8, further comprising an additional heat exchanger arranged downstream the feed-effluent heat exchanger and arranged to cool the further cooled first synthesis gas stream.

10. The methanol plant according to claim 1, further comprising a hydrogen recovery unit arranged to receive at least a portion of the off-gas stream from the methanol synthesis section and to output a hydrogen rich gas stream and a reject stream.

11. The methanol plant according to claim 1, further comprising a low pressure separator arranged to receive at least a portion of the product stream comprising methanol from the methanol synthesis section and to output a purified methanol stream and a carbon-rich stream).

12. The methanol plant according to claim 1, further comprising a methanol distillation section arranged to receive at least a portion of the product stream comprising methanol from the methanol synthesis section or at least a portion of the purified methanol stream from the low pressure separator and to output a high-purity methanol stream and a higher alcohols stream.

13. The methanol plant according to claim 12, wherein at least a portion of the heat required for the methanol distillation section is arranged to be provided from the first heat exchanger.

14. A process for producing a product stream comprising methanol, in a methanol plant according to claim 1, said process comprising the steps of:
providing the methanol plant,
supplying first feed and second feed to the CO2 shift unit, and providing a first synthesis gas stream,
supplying the first synthesis gas stream to the water removal unit and removing water from said first synthesis gas stream so as to provide a second synthesis gas stream,
supplying at least a portion of said second synthesis gas stream, and—optionally—a portion of said second feed and/or said first feed, to the methanol synthesis section, and providing a product stream comprising methanol and an off-gas stream.

15. The process according to claim 14, wherein said first feed additionally comprises up to 20%, such as up to 10% hydrocarbons.

16. The process according to claim 15, wherein said first feed additionally comprises steam.

17. A process for retrofitting a carbon dioxide-to-methanol plant, said plant comprising:
a first feed comprising CO2 to said plant, and a second feed comprising H2 to said plant;
or a combined feed comprising CO2 and H2 to said plant;
a methanol synthesis section;
wherein said retrofitting process comprises the steps of:
incorporating a CO2 shift unit, being an electrically heated Reverse Water Gas Shift unit, and a water removal unit in said plant, upstream said methanol synthesis section,
arranging at least a portion of said first feed and said second feed, or of said combined feed comprising CO2 and H2, to be supplied to the CO2 shift unit so as to provide a first synthesis gas stream,
feeding at least a portion of said first synthesis gas stream from the CO2 shift unit to the water removal unit, to remove water from said first synthesis gas stream and provide a second synthesis gas stream,
arranging at least a portion of the second synthesis gas stream to be supplied to a compressor and compressing it, so as to provide a compressed second synthesis gas stream,
arranging at least a portion of the compressed second synthesis gas stream to be supplied to the methanol synthesis section, and providing a product stream comprising methanol.

18. The process according to claim 17, wherein said carbon dioxide-to-methanol plant comprises a compressor, wherein—before the retrofitting process—said compressor is arranged to compress the combined feed, and wherein—after said retrofitting process—said compressor is arranged to compress at least a portion of the second synthesis gas stream and provide a compressed second synthesis gas stream.

19. The process according to claim 17, wherein said combined feed also comprises hydrocarbons.

20. The process according to claim 19, said combined stream is obtained by mixing a biogas with hydrogen.

21. The process according to claim 17, wherein said methanol synthesis section of said carbon dioxide-to-methanol plant remains unchanged under said retrofitting process.

22. The process according to claim 21, wherein the production of methanol from the carbon dioxide-to-methanol plant after the retrofitting process is at least 10% higher, 20% higher, 33% higher, 50% higher, 100% higher or 250% higher than the methanol production from the carbon dioxide-to-methanol plant prior to the retrofitting process, when evaluated on the flow of methanol molecules from the methanol synthesis section.

23. The process according to claim 17, wherein the methanol synthesis section comprises a recycle compressor, a heat exchanger, a flash separation vessel, and/or a cooling unit, wherein said refitting process further comprises one or more further steps of upgrading one or more of said recycle compressor, heat exchanger, flash separation vessel and cooling unit.

24. The process according to claim 17, wherein the methanol synthesis section comprises a methanol synthesis reactor, wherein said refitting process further comprises one or more further steps of reducing the catalyst loading in the methanol synthesis reactor.

25. The process according to claim 23, wherein the production of methanol from the carbon dioxide-to-methanol plant after the retrofitting process is at least the production of methanol from the carbon dioxide-to-methanol plant after the retrofitting process is at least 10% higher, 20% higher, 33% higher, 50% higher, 100% higher, 250% higher, 500% higher, 750% higher or 1000% higher than the methanol production of the conventional carbon dioxide-to-methanol plant prior to the retrofitting process when evaluated on the flow of methanol molecules from the methanol synthesis section.

26. The process according to claim 17, wherein said retrofitting process further comprises a step of arranging a hydrogen recovery unit so as to receive at least a portion of the off-gas stream from the methanol synthesis section and to output a hydrogen rich gas stream and a reject stream.
